(19) United States
(12) Patent Application Publication
(10) Pub. No.: US 2023/0138927 A1
(43) Pub. Date: May 4, 2023

(54) METHODS FOR ASSESSING THE SEVERITY OF, AND FOR INHIBITING, PRO-INFLAMMATORY RESPONSES WITH TOTAL CELL-FREE DNA

(71) Applicant: The Medical College of Wisconsin, Inc., Milwaukee, WI (US)

(72) Inventors: Michael Mitchell, Elm Grove, WI (US); Aoy Tomita Mitchell, Elm Grove, WI (US)

(73) Assignee: The Medical College of Wisconsin, Inc., Milwaukee, WI (US)

(21) Appl. No.: 17/960,904

(22) Filed: Oct. 6, 2022

Related U.S. Application Data
(63) Continuation of application No. PCT/US2021/026016, filed on Apr. 6, 2021.
(60) Provisional application No. 63/027,379, filed on May 20, 2020.
(60) Provisional application No. 63/027,402, filed on May 20, 2020.
(60) Provisional application No. 63/005,778, filed on Apr. 6, 2020.
(60) Provisional application No. 63/005,805, filed on Apr. 6, 2020.

(51) Int. Cl. C12Q 1/6883

(52) U.S. Cl. CPC C12Q 1/6883; C12Q 2600/106; C12Q 2600/118; C12Q 2600/158

(57) ABSTRACT

This invention relates to methods and compositions for assessing an amount of total cell-free DNA, such as from a subject having a pro-inflammatory response, such as a pro-inflammatory response associated with an infection, such as a COVID-19 infection. This invention also relates to compositions and methods for treating subjects having or suspected of having a pro-inflammatory response, such as a pro-inflammatory response associated with an infection, such as a COVID-19 infection. In embodiments, there is a risk of an inappropriate or detrimental pro-inflammatory response, such as in a subject with a COVID-19 infection. The methods and composition provided herein can be used to determine severity and risk of complications in a subject. The methods and composition provided herein can also be used to assess the effectiveness of a therapy in a subject, such as a COVID-19 subject.

### RELATED APPLICATIONS

This application claims the benefit of priority under 35 U.S.C. § 119 to U.S. Provisional Application No. 63/005,778, filed Apr. 6, 2020; U.S. Provisional Application No. 63/005,805, filed Apr. 6, 2020; U.S. Provisional Application No. 63,027,379, filed May 20, 2020; and U.S. Provisional Application No. 63/027,402, filed May 20, 2020, the entire contents of each of which are incorporated herein by reference.

### FEDERAL FUNDING

This invention was made with government support under grant R01 HL119747, awarded by the National Institutes of Health. The government has certain rights in this invention.

### FIELD OF THE INVENTION

This invention relates to methods and compositions for assessing an amount of total cell-free nucleic acids in a sample from a subject having a pro-inflammatory response, particularly one where the pro-inflammatory response is an inappropriate or detrimental pro-inflammatory response, such as in a subject with an infection, such as a COVID-19 infection. Such amounts can be used to determine severity and/or risk of one or more complications associated with the pro-inflammatory response. The amounts can also be used to assess response to therapy in the foregoing subjects.

This invention also relates to compositions and methods for treating subjects having or suspected of having a pro-inflammatory response, particularly one where there is a risk of an inappropriate or detrimental pro-inflammatory response, such with an infection, such as a COVID-19 infection.

### SUMMARY OF INVENTION

The present disclosure is based, at least in part, on the surprising discovery that severity and/or risk, such as of a condition and/or complications, associated with a pro-inflammatory response (e.g., one associated with an infection, such as a COVID-19 infection) is correlated with the amount of total cell-free DNA (e.g., measured as nuclear cell-free DNA). Using any one of a variety of means to quantify total cell-free DNA in a sample, the severity and/or risk of complications can be determined as well as monitored over time. Furthermore, the conditions associated with a pro-inflammatory response, such as an infection, such as COVID-19 infection, can be treated with agents that inhibit or reduce total cell-free DNA.

In one embodiment of any one of the methods provided herein, the method comprises administering one or more agents that inhibit or reduce total cell-free DNA. In one embodiment of any one of the methods provided herein, the subject is one that has been determined to have or is suspected of having the pro-inflammatory response, such as one with an infection, such as a COVID-19 infection. In another embodiment of any one of the methods provided herein, the subject is one that has a non-mild or severe pro-inflammatory response or is one at risk of one or more complications of the pro-inflammatory response.

In one embodiment, the methods can comprise steps of assessing the levels of total cf-DNA in the subject (e.g., measured as nuclear cf-DNA). These steps can be used to monitor the subject over time to assess the efficacy of the treatment and/or to identify those in need of or who would benefit from the treatment.

Provided herein are methods, compositions and kits related to such a determination and/or treatment. The methods, compositions, or kits can be any one of the methods, compositions, or kits, respectively, provided herein, including any one of those of the Examples or Figures.

In one embodiment of any one of the methods provided, the method further comprises obtaining a sample from the subject.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

In one aspect, a report or database comprising one or more of the amounts provided herein is provided.

In one aspect, any one of the methods provided herein is provided. In one embodiment of any one of the methods provided herein, the amount indicative of severity and/or risk of a complication is any one of the thresholds as described herein. In one embodiment of any one of the methods provided herein, the time for obtaining the sample is any one of the times described herein. In one embodiment of any one of the methods provided herein, the subject is any one of the subjects described herein.

In one aspect, a method of treating a subject, determining a treatment regimen for a subject, or providing information about a treatment to the subject, based on the amount of total cell-free DNA (e.g., measured as nuclear cf-DNA) or any one of the methods of analysis provided herein is provided. In one embodiment of any one of such methods, the method comprises a step of treating the subject or providing information about a treatment to the subject. In one embodiment of any one of the methods of treating, the treatment may be any one of the treatments provided herein, or otherwise known to those of ordinary skill in the art. In one embodiment of any one of the methods of treating, the treatment is for any one of the conditions provided herein. Examples of which are provided herein or otherwise known to those of ordinary skill in the art.

In any one of the methods provided herein the methods may comprise treating, determining a treatment regimen for, or providing information about a treatment to any one of the subjects provided herein.

### BRIEF DESCRIPTION OF FIGURES

The accompanying figures are not intended to be drawn to scale. The figures are illustrative only and are not required for enablement of the disclosure.

FIG. 1 illustrates an example of a computer system with which some embodiments may operate.

FIG. 2 includes two graphs and a table showing the correlation between total cell-free DNA and death, using a cutoff value of 50 ng/mL.

FIG. 3 is a graph using receiver operator characteristic (ROC) analysis on repeated measures using correlation to examine the relationship between death and total cf-DNA (whole blood and plasma). 1150 samples from 197 patients followed for at least one year following transplant were analyzed.

FIG. 4 includes two graphs and a table showing the correlation between total cf-DNA and death in pediatric samples (whole blood and plasma) following transplant.

FIG. 5 includes two graphs and a table showing the correlation between total cf-DNA and death in adult samples (whole blood and plasma) following transplant.

FIGS. 6A-6D show different experimental cutpoints (thresholds) for total cf-DNA and time to death. 50 ng/mL (FIG. 6A), 25 ng/mL (FIG. 6B), and 10 ng/mL (FIG. 6C) were examined. The results are tabulated in FIG. 6D.

FIG. 7 shows product-limit survival estimates for subjects based on total cf-DNA. The samples were taken from patients after transplant, and the time from the test to the events of death, cardiac arrest, or need for mechanical circulatory support, was examined.

FIG. 8 includes two graphs and a table showing the correlation between total cf-DNA and an event (death, cardiac arrest, or need for mechanical circulatory support).

FIG. 9 shows an analysis of three different cutoffs (thresholds): 50 ng/mL, 25 ng/mL, and 10 ng/mL.

FIG. 10 shows the correlation between nuclear cell-free DNA (ncfDNA) and severity of clinical outcomes (p<0.01 nonparametric Cusick's test).

### DETAILED DESCRIPTION OF THE INVENTION

It has been found that total cell-free DNA (e.g., nuclear cf-DNA) is correlated with tissue injury and can be used to assess and/or monitor a subject in a number of instances, such as in the transplant or other surgical context. The use of total cf-DNA can now be extended for assessing and monitoring a subject with a pro-inflammatory response, such as one associated with an infection, such as a COVID-19 infection. Furthermore, it is believed that agents that reduce or inhibit cell-free DNA (i.e., cf-DNA inhibitors) can be used to treat subjects with a pro-inflammatory response, such as one with an infection, such as a COVID-19 infection. Thus, provided herein, are methods of treating subjects having or suspected of having such an infection with a cf-DNA inhibitor, such as with those provided herein.

Described herein, are methods that can be used to assist in early identification of the severity of illness and likelihood of complications, such as progression to acute respiratory distress syndrome (ARDS) when the subject is one with an infection, such as a COVID-19 infection. Measuring circulating cell-free DNA (cf-DNA) can rapidly and effectively assist the clinician in making assessments and can save lives while greatly unburdening the health care system. In addition, as novel treatments come on line, monitoring cell-free DNA levels can be an essential measure of the effectiveness of therapy.

Cell-free DNA (cf-DNA) is found in very low concentrations in the plasma of healthy patients due to baseline cellular leakage during natural cell turnover. However, it can become very high when dying cells release DNA in much greater amounts than normal into the circulation. When optimized protocols for sample handling and qPCR analysis are used, the released cf-DNA can be measured very precisely, sensitively, quickly, and noninvasively across a wide range of concentrations as a biomarker of severity of illness using only a small peripheral blood sample that can be shipped at ambient temperature (e.g., 2 mls of blood collected by a simple peripheral blood draw). Such protocols have been tested and validated for the clinical-grade quantitative analysis of cf-DNA, as it has been applied clinically to cardiac surgical and transplant patients who are at heightened risk for not only cardiac, but multiorgan injury and failure. The utility of cf-DNA biomarker testing has been shown in over 7500 samples from 780 patients (540 cardiac transplant patients, 120 pediatric cardiac surgical patients, and 120 additional patients in two pilot studies). It has been found that cf-DNA levels begin to rise even before clinical symptoms become apparent, become higher with progression to clinically apparent illness, and are quantitatively correlated with longer term clinical outcomes. Cf-DNA levels over 50 ng/ml predicted increased likelihood of death, cardiac arrest or mechanical circulatory support within 30 days (p=0.0001, AUC=0.89, NPV=0.99), levels over 25 ng/ml predicted longer hospital length of stay (greater than 30 days) (p≤0.01), and levels over 10 ng/ml predicted presence of infections (p<0.01) that go on to require clinical treatment.

Thus, cf-DNA can be a marker for tissue injury, but its use to signal alarm and guide patient care decisions can vary depending on the clinical scenario. Organ transplant recipients are immunocompromised iatrogenically and demonstrate important similarities to COVID-19 patients in their clinical response to superinfections, ARDS, and to cytokine storm. Immunosuppressive and anti-inflammatory medications are being increasingly used to treat severe COVID-19 cases, hinting at further commonality between these two patient groups. It is believed that assessments and measures of cf-DNA can now be extended to subjects with a pro-inflammatory response, such as one with an infection associated with a pro-inflammatory response, such as a COVID-19 infection, and cf-DNA levels in these patients are expected to show very similar patterns of change in response to worsening disease and response to therapy. As described herein, a proportional increase in cf-DNA level can be indicative of increasing severity and/or presence of one or more complications in the subjects described herein. Importantly, the short (15-30 min) half-life of individual cf-DNA molecules in the patient's plasma makes the cf-DNA concentration at any given time an accurate snapshot of the current level of risk in that patient at the time of sample collection. In one embodiment, any one of the methods of treatment provided herein can be of a subject identified with the methods of assessment as provided herein. In another embodiment, any one of the methods of treatment provided herein is of a subject determined to have a cf-DNA level as provided herein.

The methods provided herein can be used to assess any subject with a pro-inflammatory response, such as one with an infection. In some embodiments, any one of the methods provided herein are used to assess a non-mild pro-inflammatory response or a non-mild infection. In some embodiments, any one of the methods provided herein are used to assess a severe pro-inflammatory response or severe infection. In some embodiments, indicators of non-mild or more severe infection can include, but are not limited to, shortness of breath, chest pain, non-healthy chest x-ray or some combination or all of the foregoing. Severe infection can also be indicated with unhealthy O₂ saturation levels. Any one of the methods provided herein, can be used for a subject with any one or more or all of the foregoing indicators.

As described herein, it is thought that cf-DNA levels (e.g., total cf-DNA) of 50 ng/ml or more will support need for intubation in patients presenting in respiratory distress. Less aggressive supportive therapy may be considered in those with lower cf-DNA levels (e.g., total cf-DNA). Early and accurate assessment can help the clinician get ahead of the disease and can assist with decision making regarding need for intensive care measures including intubation. Additionally, cf-DNA levels (e.g., total cf-DNA) would be expected to drop in response to successful therapy (generally, but not limited to, in at least near real-time). Therefore, aspects of the disclosure relate, at least in part, to methods of quantifying total cf-DNA (e.g., ncfDNA) in a sample in order to assess or determine severity and/or complication or risk associated with a pro-inflammatory response, such as an infection associated with a pro-inflammatory response (e.g., COVID-19 infection) (e.g., risk of developing ARDS, pneumonia, or death), and/or response to therapy. In some embodiments, the subject may be on mechanical support (e.g., a ventilatory and/or circulatory) and can be monitored with any one of the methods provided herein. Again, any one of such subjects can be treated with a cf-DNA inhibitor as provided herein.

As used herein, “cell-free DNA” (or “cf-DNA”) is DNA that is present outside of a cell, e.g., in the blood, plasma, serum, urine, etc. of a subject. “Total cell-free DNA” (or “total cf-DNA”) is the amount of cf-DNA present in a sample. Provided herein are methods and compositions that can be used to measure total cf-DNA which may then be used to assess the subject's risk associated with a pro-inflammatory response. Such a response can occur when a subject has an infection, such as a COVID-19 infection. As used herein, “COVID-19” refers to the novel coronavirus disease 2019 which was first identified in Wuhan, China. Any one of the methods or compositions provided herein may be used on a sample from a subject that has or is suspected of having a pro-inflammatory response that may be detrimental, such as in a subject with an infection, such as a COVID-19 infection. In some embodiments of any one of the methods provided herein, the subject is asymptomatic. In other embodiments of any one of the methods provided herein, the subject is symptomatic.

Symptoms of infections, such as COVID-19 infections can include, but are not limited to, fever, dry cough, shortness of breath (difficulty breathing), fatigue, sore throat, aches, runny nose, and the loss of smell or taste, or some combination thereof. In severe cases of infection, such as COVID-19 infection, a subject may develop pneumonia and/or acute respiratory distress syndrome (ARDS). Importantly, amounts of total cf-DNA can be used to assess or determine a risk associated with a condition (e.g., severity of the disease) in the subject. For example, COVID-19 complications include pneumonia, ARDS, etc. A severe pro-inflammatory response such as with an infection, such as COVID-19, can be fatal, and the methods and compositions provided herein can be used to assess the risk of death in subjects with such a response or infection.

As provided herein, any one of the methods can be used to assess a subject that has or is suspected of having a pro-inflammatory response, such as an infection, such as COVID-19. As used herein, “suspected of having” refers to a subject whereby a clinician believes there is a likelihood the subject has a pro-inflammatory response. For example, the subject may be suspected of having COVID-19 (e.g., a subject exhibiting symptoms of COVID-19 and/or a subject who has been in contact with another person having COVID-19). In one embodiment of any one of the methods provided herein, the subject may be one that has a complication, such as a COVID-19 complication, or that a clinician believes there is a likelihood of having a complication, such as a COVID-19 complication. In some embodiments, any one of the methods can be used to assess a subject that has had or is at risk of having a complication, such as a COVID-19 complication. Subjects may be suspected of having, determined to have had, or determined to have a likelihood or risk of having a complication (e.g., severe course of the disease, such as a COVID-19 infection) based on symptoms (and/or lack thereof). However, in some embodiments, the subject is suspected of having, determined to have had, or determined to have a likelihood or risk of having a complication, such as a COVID-19 complication, based on one or more other tests. In such an embodiment, the methods provided herein can be used to confirm such a finding or monitor such a subject for worsening or improving condition. In any one of the methods provided herein, the method may further comprise performing one or more additional tests to assess the subject's condition.

A subject may be assessed by determining or obtaining one or more amounts of total cf-DNA. An amount of total cf-DNA may be determined with experimental techniques, such as those provided elsewhere herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e., by purchasing), in some embodiments.

Because of the ability to determine amounts of cf-DNA, and the correlation with complications, risk, etc., the methods and compositions provided herein can be used to assess subjects. Thus, a risk of improving or worsening condition can be determined in such subjects. A “risk” as provided herein, refers to the presence or absence or progression of any undesirable condition in a subject, or an increased likelihood of the presence or absence or progression of such a condition. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or nonprogression) of such a condition. In one embodiment of any one of the methods provided herein, the condition is an infection associated with a pro-inflammatory response, COVID-19, pneumonia associated with the foregoing, or ARDS associated with the foregoing.

As provided herein, early detection or monitoring of complications can facilitate treatment and improve clinical outcomes. As mentioned above, any one of the methods provided can be performed on a subject that has or is suspected of having a complication, such as a COVID-19 complication. Such methods can be used to monitor a subject over time, with or without treatment. Further, such methods can aid in the selection, administration and/or monitoring of a treatment or therapy. Accordingly, the methods provided herein can be used to determine a treatment or monitoring regimen. The subject may be any one of the subjects provided herein. In one embodiment of any one of the methods provided herein, the subject is one that is on mechanical support or that is in need of mechanical support.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

Treatments include any treatment that is indicated based on the risk that is determined. In one embodiment, the treatment is an ARDS treatment. ARDS treatments include, for example, mechanical ventilation, prone positioning, sedation and medications to prevent movement, fluid management (e.g., diuretics), and extracorporeal membrane oxygenation (ECMO), although such treatments may also be applicable to any one of the subjects provided herein, and are also specifically contemplated for such subjects.

In another embodiment, the treatment can be for pneumonia. Pneumonia treatments include, for example, cough suppressants, fever reducers (e.g., aspirin, acetaminophen), and mechanical ventilation, although such treatments may also be applicable to any one of the subjects provided herein, and are also specifically contemplated for such subjects.

In another embodiment, the treatment can be an anti-viral medication, such as Remdesivir, or any other medication known in the art. Other treatments include supportive care, such as rest and hydration.

In another embodiment, the treatment can be a treatment for infection. In some embodiments, therapies for treating infection include therapies for treating a viral infection.

The treatment can include one or more inhibitors of cf-DNA (e.g., DNase I agents). An “inhibitor of cf-DNA” is any agent that reduces or inhibits the amount of cf-DNA and/or its contribution to a pro-inflammatory response. Such agents include those that degrade cf-DNA. Other agents include those that block the production of cf-DNA. Still others are those that block the pro-inflammatory activities of cf-DNA.

Examples of cf-DNA inhibitors include, but are not limited to cationic nanoparticles (Liang et al., Nat Commun. 2018; 9: 4291) and deoxyribonucleases (DNases) (Cagliani et al., J of Surg Res., May 2020 (249): 104-113). DNases are enzymes that catalyze the hydrolytic cleavage of phosphodiester linkages in an DNA backbone. DNase I has been shown to increase survival of hemorrhaged mice having elevated levels of cf-DNA (Cagliani et al.). Examples of DNases include, but are not limited to, DNase I (e.g., recombinant human DNase I (rhDNase I) or bovine pancreatic DNase I), analogues of DNase I (such as, e.g., DNase X, DNase gamma, and DNAS1L2), DNase II (e.g., DNase II-alpha, DNase II-beta), phosphodiesterase I, lactoferrin, and acetylcholinesterase. In one embodiment of any one of the methods provided herein, DNase I (e.g., PULMOZYME™) is administered. DNase I cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

Examples of cf-DNA inhibitors also include cationic polymers, which can neutralize cf-DNA. Other examples of cf-DNA inhibitors include inhibitors of the signaling pathway, including downstream, and blocking receptors for cf-DNA such as TLR9 or preventing TLR9 activation. Cell-free DNA is a stress signal in the danger associated molecular pattern (DAMP) pathway. Cf-DNA can activate Toll-like receptor 9 (TLR9) to secrete inflammatory cytokines. Blocking this TLR9 activation can potentially stop cytokine release/inflammatory response by stopping this immune response pathway. Thus, cf-DNA inhibitors include those that can block this activation (e.g., antibodies). Examples of downstream cf-DNA inhibitors include, but are not limited to, inhibitors of NLRP3 and IL-1. NLRP3 inhibitors include, but are not limited to, Cl− channel inhibitors (flufenamic acid, IAA94, DIDS, NPPB, etc.), G5, MCC950, JC124, colchicine, CY-09, ketone metabolite beta-hydroxubutyrate (BHB), a type I interferon, resveratrol, arglabin, CB2R, glybenclamide, isoliquiritigenin, Z-VAD-FMK, and microRNA-223. IL-1 inhibitors include, but are not limited to, interleukin-1 receptor antagonists (e.g., IL-1ra) anti-IL-1 receptor monoclonal antibodies (e.g., canakinumab); IL-1 binding proteins (e.g., soluble IL-1 receptors (e.g., U.S. Pat. Nos. 5,492,888, 5,488,032, and 5,464,937, 5,319,071, and 5,180,812, the disclosures of which agents are hereby incorporated by reference herein)); anti-IL-1 monoclonal antibodies (e.g., anakinra, rilonacept); IL-1 receptor accessory proteins and antibodies thereto (e.g., WO 96/23067 and WO 99/37773, the disclosures of which agents are hereby incorporated by reference herein); inhibitors of interleukin-1 beta converting enzyme (ICE) or caspase I (e.g., N-benzyloxycarbonyl-Val-Ala-Asp-fluoromethylketone (z-VAD.FMK), acetyl-Tyr-Val-Ala-Asp-chloromethylketone, N-benzyloxycarbonyl-Asp-Glu-Val-Asp-fluoromethylketone), interleukin-1 beta protease inhibitors; and other compounds and proteins which block in vivo synthesis or extracellular release of IL-1.

In another embodiment, the therapy or treatment can comprise use of an inhibitor of IL-6 or the IL-6 receptor, such as human monoclonal antibodies against IL-6 receptor (e.g., tocilizumab (RoActemra, Roche) and sarilumab (Kevzara, Sanofi)).

As another example, cf-DNA inhibitors can include an agent that prevents release of cf-DNA through NETosis and/or blocks platelet formation or activation. Without being bound by any particular theory, apart from apoptotic and necrotic cell death, DNA release mechanisms include neutrophil extracellular trap release (NETosis). Platelet activation can trigger NETosis (which can increase cell-free DNA). Thus, agents that prevent platelet activation or formation and/or NETosis, can also be used as a cf-DNA inhibitor as provided herein. For example, such agents can include aspirin, heparin, etc.

In another embodiment, downstream decrease in cell-free DNA levels can be achieved by treatment the includes prone positioning and/or Remdesivir.

In another embodiment, the treatment can comprise any treatment whereby cf-DNA is removed from the blood of the subject. Such treating can include with a device that includes a filter to reduce the amount of cell-free DNA in the subject. In one embodiment of any one of the methods provided herein, the treatment comprises apheresis and/or an extracorporeal filter (e.g., CytoSorb®) as part of an ECMO treatment for any one of the subjects provided herein. In one embodiment, the treatment comprises use of an extracorporeal filter (e.g., CytoSorb®) as part of an ECMO treatment or cardiopulmonary bypass for any one of the subjects provided herein.

The cf-DNA inhibitors can be administered in effective amounts. “Amount effective” in the context of a composition for administration to a subject as provided herein refers to an amount of the composition or dosage form that produces one or more desired results in the subject, for example, the prevention of any undesired immune response. An amount effect can also be an amount that results in a reduction or elimination of a pro-inflammatory immune response and/or a decrease in the level of cf-DNA in the subject, etc An amount effect can also be an amount that results in a desired therapeutic endpoint or a desired therapeutic result. The achievement of any of the foregoing can be monitored by routine methods. The amount effective can be for in vitro or in vivo purposes. For in vivo purposes, the amount can be one that a clinician would believe may have a clinical benefit for a subject and will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. In any one of the methods provided herein, the composition(s) administered may be in any one of the amounts effective as provided herein. In one embodiment of any one of the methods provided herein, the subject can be treated with a cf-DNA inhibitor when the amount of cf-DNA is above any one of the thresholds provided herein. In one embodiment of any one of the methods provided herein, the subject is treated with any one of the devices as provided herein including any one of the mechanical support devices provided herein that can also include a filter to reduce the level of cf-DNA in the subject.

Other therapies are known to those of ordinary skill in the art. In some embodiments of any one of the methods provided herein, the treatment comprises one or more of the treatments described herein. The methods provided herein can include the administration of one or more additional therapies, treatments, etc.

In another embodiment, the additional treatment is an ARDS treatment. ARDS treatments include, for example, mechanical ventilation, prone positioning, sedation and medications to prevent movement, fluid management (e.g., diuretics), and extracorporeal membrane oxygenation (ECMO), although such treatments may also be applicable to any one of the subjects provided herein, and are also specifically contemplated for such subjects.

In another embodiment, the treatment can be for pneumonia. Pneumonia treatments include, for example, cough suppressants, fever reducers (e.g., aspirin, acetaminophen), and mechanical ventilation, although such treatments may also be applicable to any one of the subjects provided herein, and are also specifically contemplated for such subjects.

In another embodiment, the treatment can be an anti-viral medication, such as Remdesivir, or any other medication known in the art. Other treatments include supportive care, such as rest and hydration.

In another embodiment, the treatment can be a treatment for infection. In some embodiments, therapies for treating infection include therapies for treating a viral infection.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

The methods provided herein can be used to treat any subject provided herein, such as one with an infection, such as a COVID-19 infection. In some embodiments, any one of the methods provided herein are used to treat a non-mild pro-inflammatory response or a non-mild infection. In some embodiments, any one of the methods provided herein are used to treat a severe pro-inflammatory response or severe infection. In some embodiments, indicators of non-mild or more severe infection can include, but are not limited to, shortness of breath, chest pain, non-healthy chest x-ray or some combination or all of the foregoing. Severe infection can also be indicated with unhealthy O₂ saturation levels. Any one of the methods provided herein, can be used for a subject with any one or more or all of the foregoing indicators.

The methods provided herein can include a step of assessing the subject. Alternatively, the methods provided herein can be performed on a subject assessed or identified as provided herein.

The treatment and clinical course may be determined based on the subject's condition as determined as provided herein and/or the subject's associated expected outcome. For example, if the amount of total cf-DNA is 10 ng/mL or greater, 25 ng/mL or greater, or 50 ng/mL or greater, the subject may be treated with, or provided information related thereto, a therapy, such as those described herein. As another example, if the amount of nuclear cell-free DNA is or 10 ng/mL or greater, 25 ng/mL or greater, or 50 ng/mL or greater, the subject may be treated with, or provided information related thereto, a therapy, such as those described herein.

A monitoring regimen may also be determined based on the subject's condition as determined as provided herein and/or the subject's associated expected outcome. “Determining a monitoring regimen”, as used herein, refers to determining a course of action to monitor a condition in the subject over time. In one embodiment of any one of the methods provided herein, determining a monitoring regimen includes determining an appropriate course of action for determining the amount of total cf-DNA in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of total cf-DNA.

In some embodiments of any one of the methods provided herein, the total cf-DNA may be measured as soon as the subject exhibits one or more symptoms or has or is expected to have a condition or complication as described herein. In other embodiments, the total cf-DNA may be quantified within 1, 2, 3, 4, 5, 6, 7, 8, 9, 10, 11, 12, 13, 14, or more days after exhibiting one or more symptoms, conditions, or complications described herein. In some embodiments of any one of the methods provided herein, the total cf-DNA may be measured after the subject has come into contact with one or more people having COVID-19. In other embodiments, the total cf-DNA may be quantified within 1, 2, 3, 4, 5, 6, 7, 8, 9, 10, 11, 12, 13, 14, or more days after the subject has come into contact with one or more people having COVID-19.

In order to monitor the subject's total cf-DNA levels, samples may be taken thrice daily, twice daily, daily, every other day, every second day, every third day, every fourth day, every fifth day, every sixth day, weekly, every other week, every second week, monthly, or at more frequent intervals for up to 1 week, 2 weeks, 3 weeks, 1 month, 2 months, 3 months, or longer. A clinician may determine that a subject should undergo more frequent sampling if the subject's total cf-DNA is found to increase between time points. If a subject is found to have decreasing levels of total cf-DNA between time points, a clinician may determine that less frequent sampling is sufficient. Timing and/or frequency of monitoring may also be determined by a comparison to one or more threshold values. For example, if the amount of total cf-DNA is equal to or greater than 50 ng/mL, 25 ng/mL, or 10 ng/mL (or any one of the thresholds provided herein) and/or is increasing, more frequent sampling may be needed, whereas, if the amount of total cf-DNA is less than 50 ng/mL, 25 ng/mL, or 10 ng/mL (or any one of the thresholds provided herein), and/or is not increasing, less frequent sampling may be required. For example, if the amount of ncf-DNA is equal to or greater than 50 ng/mL, 25 ng/mL, or 10 ng/mL (or any one of the thresholds provided herein) and/or is increasing, more frequent sampling may be needed, whereas, if the amount of ncf-DNA is less than 50 ng/mL, 25 ng/mL, or 10 ng/mL (or any one of the thresholds provided herein), and/or is not increasing, less frequent sampling may be required. Generally, subjects with higher or increasing amounts of total cf-DNA require closer monitoring and more frequent sampling. In some embodiments of any one of the methods provided herein, each amount and time point may be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of total cf-DNA in a sample. In some embodiments, the report provides amounts of total cf-DNA in samples from a subject over time.

In some embodiments, the amounts are in or entered into a database. In one aspect, a database with such values is provided. From the amount(s), a clinician may assess the need for a treatment or monitoring of a subject. Accordingly, in any one of the methods provided herein, the method can include assessing the amount of cf-DNA in the subject at more than one point in time. Such assessing can be performed with any one of the methods or compositions provided herein.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values. Generally, unless otherwise provided, the amounts provided herein represent the total cf-DNA in a sample.

In some embodiments, any one of the methods provided herein can comprise comparing an amount to a threshold value, or to one or more prior amounts, to identify a subject at increased or decreased risk. In some embodiments of any one of the methods provided herein, a subject having an increased amount of total nucleic acids compared to a threshold value, or to one or more prior amounts, is identified as being at increased risk. In some embodiments of any one of the methods provided herein, a subject having a decreased or similar amount of total cf-DNA compared to a threshold value, or to one or more prior amounts, is identified as being at decreased or not increased risk.

“Threshold” or “threshold value” or “cutpoint”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold value of any one of the methods provided herein, can be any one of the threshold values provided herein, such as in the Examples or Figures.

The threshold values provided herein can be used to determine a risk, such as of one or more complications in a subject. Accordingly, if the amount of total cf-DNA measured is equal to or greater than 8, 9, 10, 11, 12, 13, 14, 15, 16, 17, 18, 19, 20, 25, 30, 35, 40, 45, 50, 55, 60, 65, 70, 75 or 80 ng/mL, then the subject may be determined to be at increased risk, such as of a complication. For example, an amount equal to or greater than 50 ng/mL may be indicative of near-term severe clinical progression, such as including the need for intubation. The determination can be done based on any one of the comparisons as provided herein with or without other indicators of such a complication. In some embodiments, if the amount of ncf-DNA measured is equal to or greater than 8, 9, 10, 11, 12, 13, 14, 15, 16, 17, 18, 19, 20, 25, 30, 35, 40, 45, 50, 55, 60, 65, 70, 75 or 80 ng/mL, then the subject may be determined to be at increased risk, such as of a complication.

The threshold values can also be used for comparisons to make treatment and/or monitoring decisions. For example, if the amount of total cf-DNA is greater than one of the thresholds provided herein and/or increasing over time, further monitoring may be indicated. As a further example, if the amount is greater than any one of the thresholds provided herein, treatment of the subject may be indicated. If the amount is greater than any one of the thresholds provided herein, additional testing of the subject, such as with a chest x-ray and/or computerized tomography (CT) may be indicated.

Accordingly, any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate. The type of additional test(s) will depend upon the condition of the subject and/or is well within the determination of the skilled artisan.

Exemplary additional tests for subjects provided herein, such as one suspected of ARDS include, but are not limited to, chest x-rays, computerized tomography (CT), lab tests, electrocardiograms, an echocardiograms.

Exemplary additional tests for subjects provided herein, such as one suspected of pneumonia include, but are not limited to, blood tests, chest x-rays, pulse oximetry, sputum tests, CT scans, and pleural fluid culture.

As another example, an additional test may be assessing the level of IL-6.

The amount of total cf-DNA may be determined by a number of methods. In some embodiments such a method is a sequencing-based method. Total cf-DNA may be analyzed using any suitable next generation or high-throughput sequencing technique. In one embodiment, any one of the methods for determining total cf-DNA may be any one of the methods of U.S. Publication No. 2015-0086477-A1, and such methods are incorporated herein by reference in their entirety. An amount of total cf-DNA may also be determined by a MOMA assay. In one embodiment, any one of the methods for determining total cf-DNA may be any one of the methods of PCT Publication No. WO 2016/176662 A1, and such methods are incorporated herein by reference in their entirety.

In some embodiments of any one of the methods provided herein, the method is an amplification-based quantitative assay, such as whereby nucleic acids are amplified and the amounts of the nucleic acids can be determined. Such assays include those whereby nucleic acids are amplified with the primers as described herein, or otherwise known in the art, and quantified. Such assays include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-stranded DNA-specific dye SYBR® Green (Molecular Probes). SYBR® Green is a dye that binds the minor groove of double-stranded DNA. When SYBR® Green dye binds to a double-stranded DNA, the fluorescence intensity increases. As more double-stranded amplicons are produced, SYBR® Green dye signal will increase.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl₂, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 50004 which may depend on the length and number of PCR products produced in a PCR reaction.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype.

In some embodiments of any one of the methods provided herein, the total cell-free DNA is determined with TAQMAN™ Real-time PCR using RNase P as a target. Other methods for determining total cell-free DNA in a sample are known in the art.

As another example, an amount of ncfDNA may be determined by a number of methods. In some embodiments, such a method is a sequencing-based method, such as multiplexed allele-specific quantitative PCR.

Any one of the methods provided herein can comprise extracting nucleic acids, such as total cell-free DNA, from a sample obtained from a subject. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such an amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

As used herein, the sample from a subject can be a biological sample. Examples of such biological samples include whole blood, plasma, serum, saliva, urine, etc. In some embodiments, addition of further nucleic acids, e.g., a standard, to the sample can be performed.

In another aspect, compositions and kits comprising one or more primer pairs as provided herein are provided. Other reagents for performing an assay, such as a PCR assay, may also be included in the composition or kit.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—Examples of Computer-Implemented Embodiments

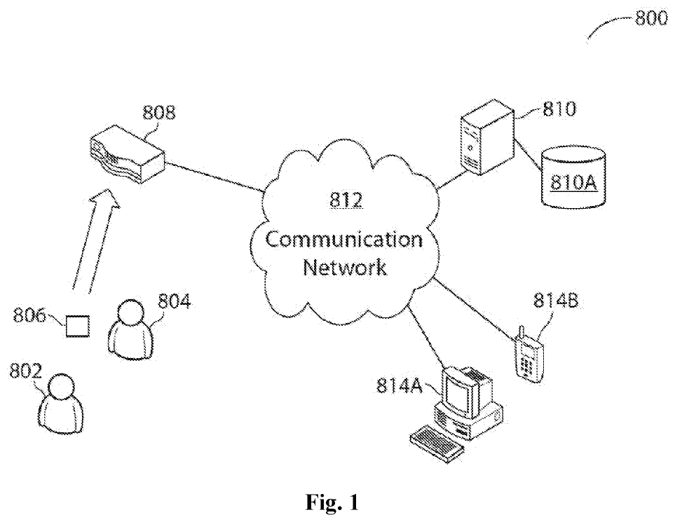

In some embodiments, the diagnostic techniques described above may be implemented via one or more computing devices executing one or more software facilities to analyze samples for a subject over time, measure nucleic acids (such as cell-free DNA) in the samples, and produce a diagnostic result based on one or more of the samples. FIG. 1 illustrates an example of a computer system with which some embodiments may operate, though it should be appreciated that embodiments are not limited to operating with a system of the type illustrated in FIG. 1.

The computer system of FIG. 1 includes a subject 802 and a clinician 804 that may obtain a sample 806 from the subject 806. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 808 so as to determine (including estimate) a total amount of nucleic acids (such as cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable foiin of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time to one or more thresholds. For example, the diagnostic facility may determine a risk to the subject 802 of a condition by comparing a total amount of nucleic acids (such as cell-free DNA) for one or more samples 806 to a threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

### Example 2—Total Cell-Free DNA (Cf-DNA) Test: Multi-Center Prospective Blinded Study

A multi-center prospective blinded study was undertaken to investigate the value of cf-DNA non-invasive clinical monitoring. 241 patients (aged 8 days to 73 years) were recruited from seven different sites. Of the patients, 146 were pediatric patients, and 95 were adults. The patients were longitudinally followed for at least one year. Samples were collected during routine catheterizations, hospital admissions, and events. In total, 2537 samples were analyzed in a blinded fashion. The relationship between total cf-DNA and death was analyzed. In all, 197 patients with 1150 samples were used for the analysis. There were 21 deaths over the study period. The mean cf-DNA values are shown below:

| | Death | Healthy | |
|---|---|---|---|
| cfDNA | 73.93 | 8.31 | p = 0.004 |
| | (5.17-777.08) | (0.07-1395.80) | |
| | ng/ml | ng/ml | |

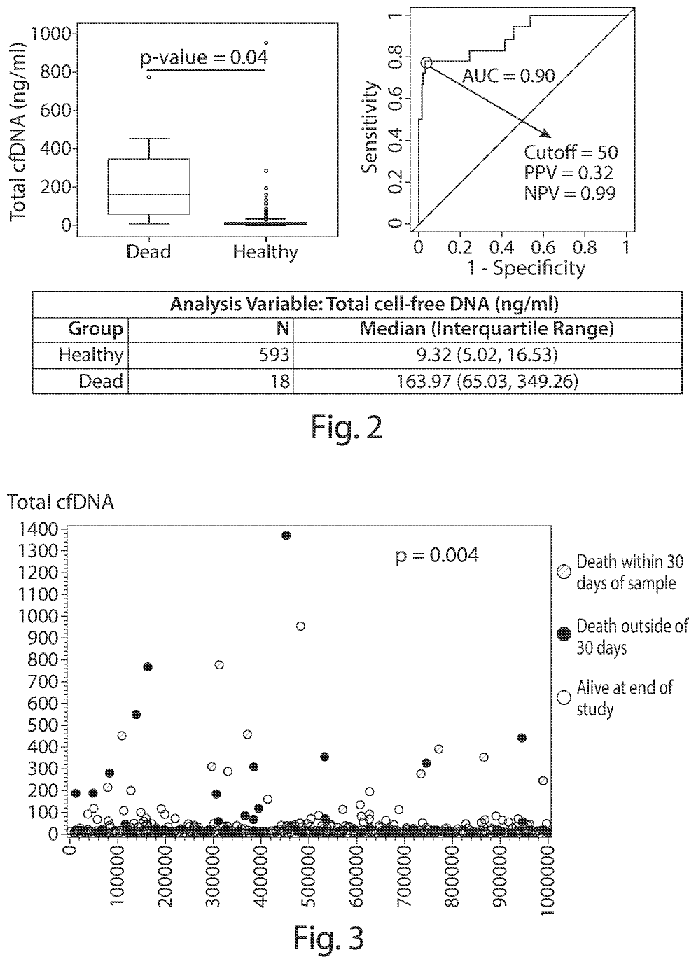

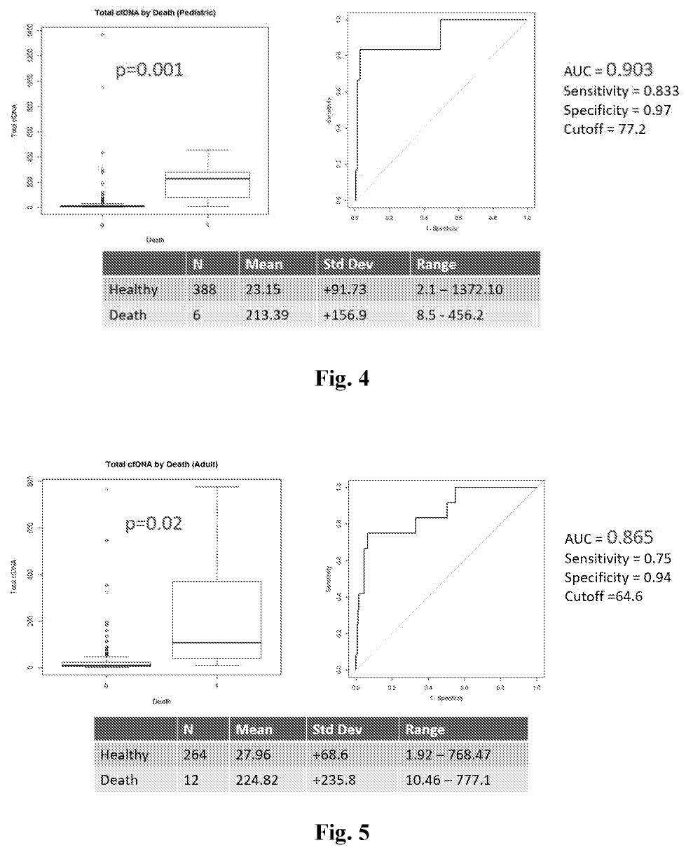

Graphs of the analysis using 50 ng/mL as the cutoff (threshold) are shown in FIG. 2. Total cf-DNA was found to predict clinical outcomes (death) as shown in FIG. 3. Whole blood and plasma samples were analyzed using ROC on repeated measures using correlation. The data was then examined for pediatric patients (FIG. 4) and adult patients (FIG. 5). The “healthy” group included samples not related to death (e.g., samples drawn more than 30 days before death) as well as those who did not die. Samples taken from patients within 7 days post-transplant were excluded from the analysis.

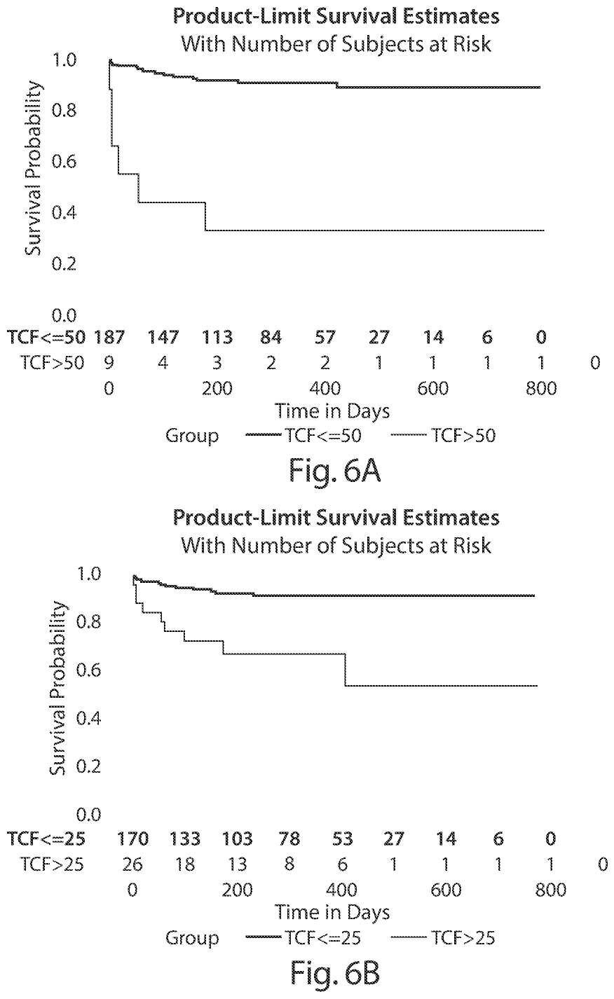

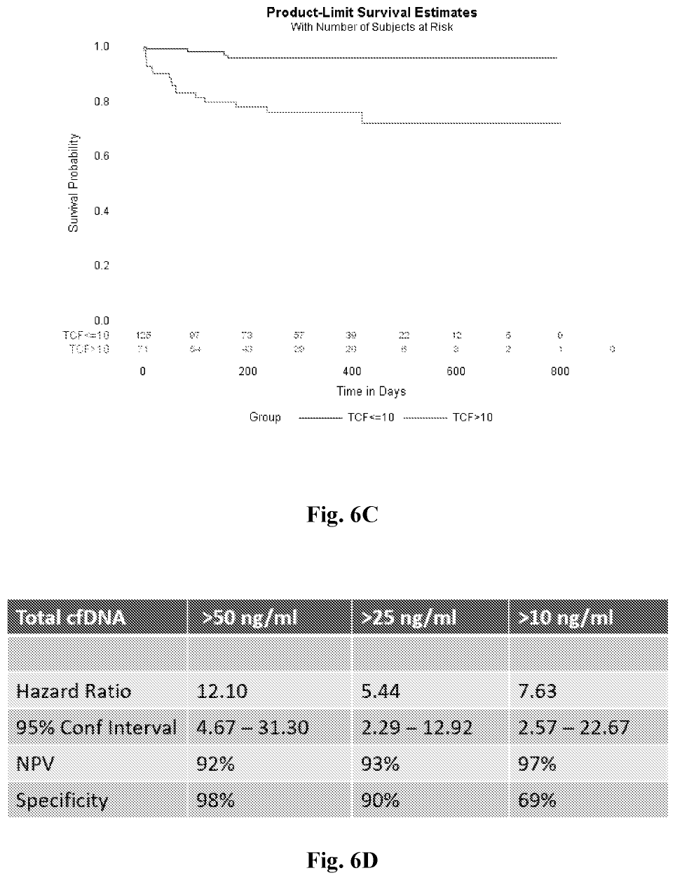

Cutoff values of 50 ng/ml, 25 ng/ml, and 10 n/ml were used to generate receiver operating characteristic (ROC) curves, which are shown in FIGS. 6A-6C. Data was graphed over time post-transplant. As can be seen in the table summarizing the results (FIG. 6D), the greatest specificity was observed with 50 ng/mL was used as the cutoff.

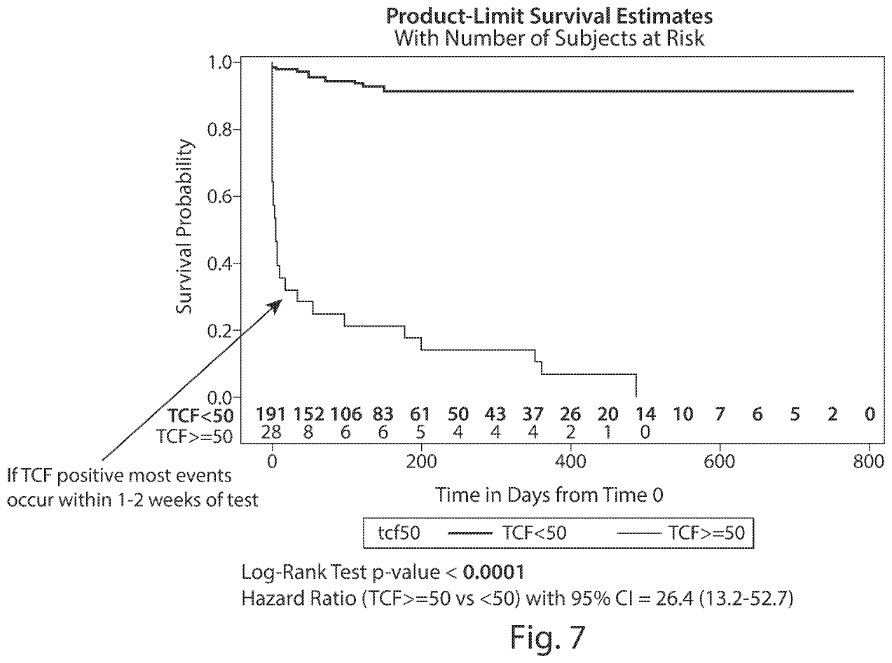

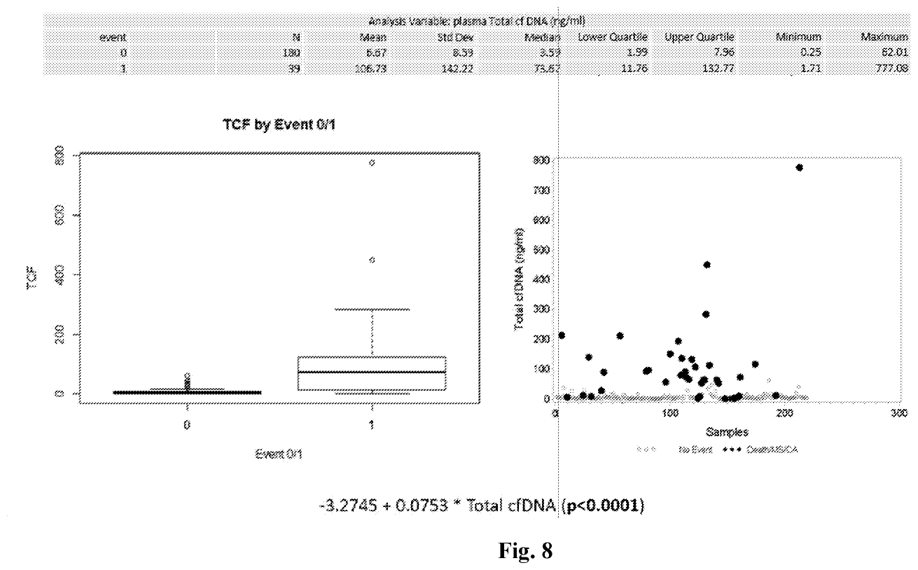

The data was analyzed for total cf-DNA and any event (death, cardiac arrest, or need for mechanical circulatory support). As can be seen in FIGS. 7-8, if the total cf-DNA is positive, most events occurred within 1-2 weeks of the test. The data is also presented in the table below (TCF=total cf-DNA):

| TCF50 | | | |
|---|---|---|---|
| Frequency | | | |
| Row Pct | Event | | |
| Col Pct | No Event | Death/MS/Cardiac Arrest | Total |
| TCF < 50 | 179 | 12 | 191 |
| | 93.72 | 6.28 | |
| | 99.44 | 30.77 | |
| TCF >= 50 | 1 | 27 | 28 |
| | 3.57 | 96.43 | |
| | 0.56 | 69.23 | |
| Total | 180 | 39 | 219 |

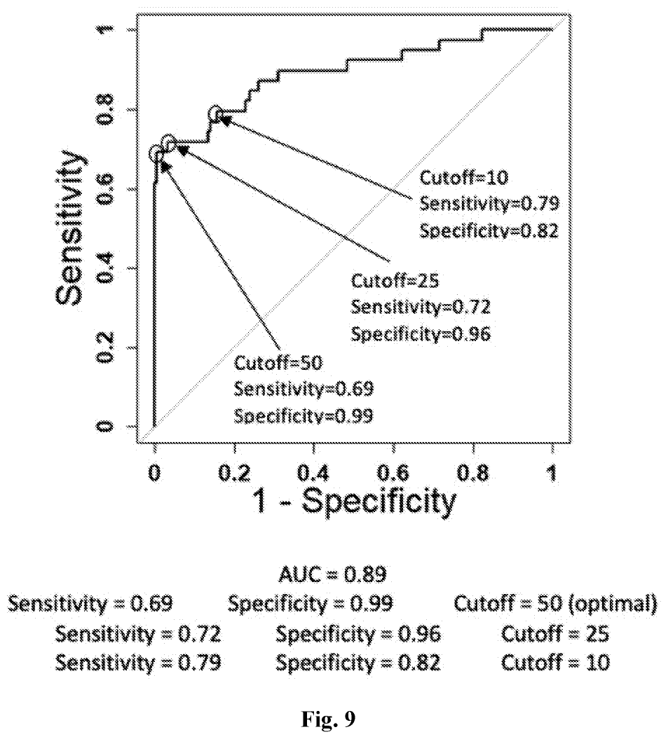

FIG. 9 shows an analysis of the different candidate cutoffs: 10 ng/ml, 25 ng/ml, and 50 ng/ml. As was demonstrated earlier, the 50 ng/ml cutoff provides the greatest specificity.

### Example 3—Total Cell-Free DNA (Cf-DNA) as Predictor of Severity of Illness and Outcome in COVID-19: Multi-Center Prospective Blinded Study

A blinded retrospective study was undertaken to investigate the use of cell-free DNA (cf-DNA) to predict the severity of illness and outcome in COVID-19. Thirty-two plasma samples (0.5 mL) were obtained from 19 patients hospitalized who had tested positive for COVID-19. Samples were extracted for cell-free DNA and quantified by total nuclear cell-free DNA (ncfDNA, ng/ml plasma) as described previously (North et al., PLoS One, 2020, Jan. 13:15(1): e0227385).

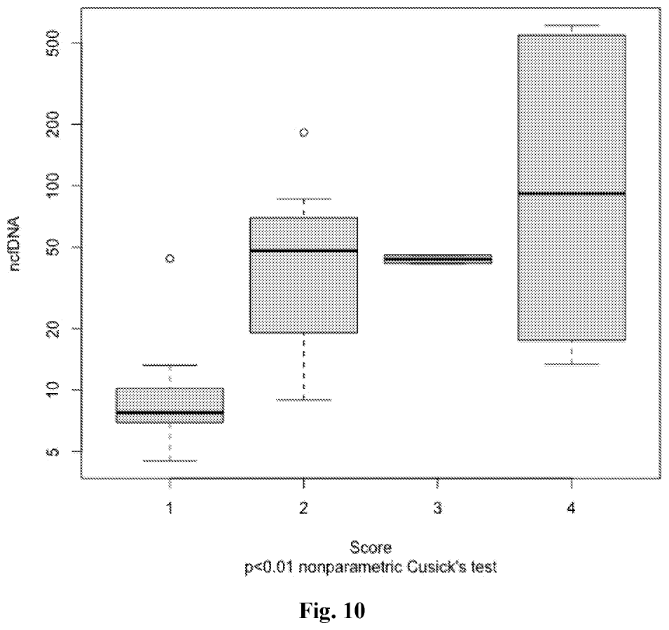

Nuclear cfDNA was found to be significantly associated with severity of clinical outcomes (p<0.01 nonparametric Cusick's test) (FIG. 10). Nuclear cfDNA was highly predictive of which subjects would go on to develop severe global symptoms.

Nuclear cfDNA was predictive of patients who would go on to die or develop severe illness. Also found was that therapies consistently reduced nuclear cell free DNA. Patients who died had elevated nuclear cfDNA. All of the surviving patients exhibited improvement in their cfDNA levels.

1. A method of assessing a sample from a subject having or suspected of having condition, such as an infection, associated with a pro-inflammatory response (e.g., a COVID-19 subject), the method comprising:
(a) determining an amount of total cf-DNA in a sample from the subject; and
(b) optionally, reporting and/or recording the amount of total cf-DNA.

2. The method of claim 1, wherein the subject has, is suspected of having, has had, or is at risk of having:
(a) one or more complications;
(b) pneumonia or acute respiratory distress syndrome; or
(c) a significantly increased risk of death relative to a healthy subject without the condition or relative to a subject with a mild case of the pro-inflammatory response or condition.

3-7. (canceled)

8. The method of claim 1, wherein the method further comprises:
(c) comparing the amount of total cf-DNA to a threshold total cf-DNA value or at least one prior total cf-DNA amount.

9. The method of claim 8, wherein the method further comprises:
(d) determining that the subject has, or as being at increased risk of having one or more complications of the condition or of death based on the determined amount of total cf-DNA compared to the threshold total cf-DNA value and/or at least one prior total cf-DNA amount.

10. A method of assessing a subject having or suspected of having condition associated with a pro-inflammatory response (e.g., a COVID-19 subject), the method comprising:
(a) obtaining an amount of total cf-DNA in a sample from the subject, wherein the subject has, is suspected of having, has had, or is at risk of having one or more complications of the condition or of death;
(b) comparing the amount of total cf-DNA to a threshold total cf-DNA value and/or at least one prior total cf-DNA amount; and
(c) optionally, determining a treatment or monitoring regimen for the subject based on the determined amount of total cf-DNA compared to the threshold total cf-DNA value and/or at least one prior total cf-DNA amount.

11. The method of claim 10, wherein the method further comprises classifying the subject as having or as being at increased risk of having one or more complications of the condition or of death based on the determined amount of total cf-DNA compared to the threshold total cf-DNA value and/or at least one prior total cf-DNA amount.

12. (canceled)

13. The method of claim 1, wherein one or more additional test(s) prior applied to the subject was performed or the method further comprises performing one or more additional test(s) on the subject.

14. The method of claim 1, wherein the total cf-DNA amount is provided in a report.

15-17. (canceled)

18. The method of claim 1, wherein the amount of total cf-DNA is determined or obtained using an amplification-based quantification assay or using sequencing.

19-20. (canceled)

21. The method of claim 1, wherein:
(a) an amount of total cf-DNA that is greater than the threshold value and/or is increased relative to the amount from an earlier time point represents an increased or increasing risk; or
(b) an amount of total cf-DNA that is lower than the threshold value and/or is decreased relative to the amount from an earlier time point represents a decreased or decreasing risk.

22. (canceled)

23. The method of claim 10, wherein the determining a monitoring regimen comprises determining the amount of total cf-DNA in the subject over time or at a subsequent point in time, or suggesting such monitoring to the subject.

24. (canceled)

25. The method of claim 10, wherein the time between samples is decreased if the amount of total cf-DNA is increased relative to the threshold or an amount from an earlier time point.

26-28. (canceled)

29. The method of claim 10, wherein the determining a monitoring regimen comprises using or suggesting the use of one or more additional test(s) to assess the subject, selecting or suggesting a treatment for the subject, providing information about a treatment to the subject and/or treating the subject.

30-33. (canceled)

34. The method of claim 10, wherein the subject has, is, or will be treated with a therapy, and the method is one to assess the efficacy of the therapy or treatment and/or to determine a treatment regimen.

35. (canceled)

36. A method of treating a subject having or suspected of having condition, such as an infection, associated with a pro-inflammatory response (e.g., a COVID-19 subject), the method comprising administering an effective amount of a cf-DNA inhibitor to the subject or comprising treating the subject such that the amount of cf-DNA is decreased.

37. The method of claim 36, wherein the cf-DNA inhibitor is/comprises cationic nanoparticles, cationic polymers, a DNase, or an inhibitor of TLR9 activation.

38. (canceled)

39. The method of claim 36, wherein the treating comprises apheresis and/or use of an extracorporeal filter, such as an extracorporeal cytokine adsorber (e.g., CytoSorb®).

40-41. (canceled)

42. The method of claim 36, wherein the method further comprises administering an additional therapy or treatment to the subject.

43-44. (canceled)

45. The method of claim 36, wherein the subject:
(a) is one with a non-mild pro-inflammatory response;
(b) is one with a severe-pro-inflammatory response;
(c) has or is at risk of having one or more complications of the condition;
(d) has or is at risk of having pneumonia or ARDS;
(e) is at increased risk of death relative to a healthy subject without the condition or relative to a subject with a mild case of the pro-inflammatory response or condition;
(f) is on mechanical support or is in need of mechanical support; or
(g) is one with, including one who was determined to have, an increased level of total cf-DNA.

46-62. (canceled)

63. The method of claim 1, wherein the threshold is 10 ng/mL or greater, 25 ng/mL or greater, or 50 ng/mL or greater.

64-66. (canceled)
